(19) United States
(12) Patent Application Publication
(10) Pub. No.: US 2020/0211687 A1
(43) Pub. Date: Jul. 2, 2020

(54) MEDICAL PRESCRIPTION MANAGEMENT SYSTEM AND METHOD

(71) Applicant: Medipick Pty Ltd, Brisbane, Queensland (AU)

(72) Inventor: Jarrod Sierocki, Brisbane (AU)

(21) Appl. No.: 16/611,449

(22) PCT Filed: May 9, 2018
(86) PCT No.: PCT/AU2018/050426

§ 371 Date: Nov. 6, 2019

(30) Foreign Application Priority Data
May 9, 2017 (AU) 2017901717

(51) Int. Cl. G16H 20/10; G16H 10/60; G16H 40/20; G16H 15/00; G16H 50/70; G06F 16/245; H04L 29/06

(52) U.S. Cl. CPC G16H 20/10; G16H 10/60; G16H 40/20; H04L 63/083; G16H 50/70; G06F 16/245; G16H 15/00

(57) ABSTRACT

The present invention relates to a system and method for the management of medical prescriptions. The system includes a data interface through which a medical practitioner can interact with the system to at least input and/or manage at least one medical prescription of a patient and at least one remote server for at least receiving the at least one medical prescription of the patient from the data interface and transmitting the at least one medical prescription to a hospital dispensary for filling.

### TECHNICAL FIELD

The present invention relates to a system and method for the management of medical prescriptions in a hospital environment.

### BACKGROUND

A medical prescription is generally a handwritten order from a physician or other qualified medical practitioner authorizing the dispensing of a prescription drug from a pharmacist.

In a hospital setting, conventional practice is for the physician or other qualified medical practitioner to issue the prescription, which is then transmitted by hand, faxed or emailed to a hospital dispensary for filling and collection.

A problem associated with conventional practice is that prescriptions are often incorrectly filled partly due to the fact that they are commonly handwritten by the physician or qualified medical practitioner. This can sometimes lead to the prescription being illegible or incorrectly read and a patient being provided a prescription drug with the wrong dosage regime, or, in the worst case scenario, being prescribed the incorrect drug.

Another problem associated with conventional practice is that, in some instances, there is a significant delay in the transmission of a prescription to a hospital dispensary, which can, in turn, delay the filling of the prescription and ultimately the administration of the prescription drugs to the patient. Indeed, in a worst case scenario, the prescription may be lost or misplaced on its way to the hospital dispensary or even prior to being filled in the hospital dispensary. Such delays can mean the difference between a speedy patient recovery and a prolonged stay in the hospital or even a fatality.

### SUMMARY OF INVENTION

Embodiments of the present invention are directed to a system and a method for the management of medical prescriptions, which may at least partially overcome at least one of the abovementioned problems or provide the consumer with a useful or commercial choice.

According to a first aspect of the present invention, there is provided a system for medical prescription management, said system including:

data interface through which a medical practitioner can interact with the system to at least input and/or manage at least one medical prescription of a patient; and

at least one remote server for at least receiving the at least one medical prescription of the patient from the data interface and transmitting the at least one medical prescription to a hospital dispensary for filling.

The data interface may be of any suitable form to enable the medical practitioner to interact with the system and at least input and/or manage the at least one medical prescription of a patient. Preferably, the data interface includes a user interface through which the medical practitioner may enter data, such as, e.g., patient details, prescription details, location details (i.e., which ward or department) and/or recipient details (i.e., who is to receive the filled prescription from the hospital dispensary).

In some embodiments, the data interface may be in the form of an external processing device, including one or more processors and a memory, for receiving input from the medical practitioner and transmitting and receiving information to and from the at least one remote server. For example, the data interface may be in the form of a computer located in a ward or department of the hospital of the medical practitioner.

In other embodiments, the data interface may be configured to be hand-held by the medical practitioner.

In one such embodiment, the data interface may be in the form of a mobile device, such as, e.g., a smart phone, a tablet or a smart watch.

In one such embodiment, the system may further include software in the form of an application (i.e., an app) configured to be run on the mobile device.

The software may preferably be interactive.

For example, the software may enable the medical practitioner to select a desired patient or even input a new patient.

Generally, the software may enable the medical practitioner to search existing patients for a desired patient. For example, in some embodiments, when searching and/or selecting a desired patient, the software may retrieve relevant patient records from the at least one remote server and display the relevant patient records for the medical practitioner's perusal and/or selection.

The software may retrieve and display the selected patient's details, including but not limited to, for example, age, gender, address details, healthcare system details (i.e., medicare number and expiry date) and/or private healthcare details (i.e., policy number).

The software may preferably retrieve and display a patient's existing prescription(s). As before, the software may retrieve the existing prescription for the selected patient from the at least one remote server. In some embodiments, the software may enable the medical practitioner to manage the selected patient's existing prescription(s). For example, the medical practitioner may alter or cancel an existing prescription. If altered, the medical practitioner may change the dosage of a previously prescribed prescription medication. Similarly, the medical practitioner may alter the existing prescription to added or reduce one or more repeats, for example.

In some embodiments in which the patient has been previously prescribed a restricted prescription medication, the software may additionally retrieve and display an authorisation number or code or authorisation approval number or code.

As indicated above, the software may preferably enable the medical practitioner to input a new prescription. Generally, the software may enable the medical practitioner to search and/or select from dispensary records a desired prescription medication. For example, in some embodiments, when searching and/or selecting a desired prescription medication, the software may retrieve relevant prescription medication information from the at least one remote server and display the relevant prescription medication information for the medical practitioner's perusal and/or selection. The relevant prescription medication information may include but not be limited to current stock of the prescription medication in the hospital dispensary, a brief description of the prescription medication and/or dosages of the prescription medication available.

Once the medical practitioner has interacted with the system to at least input and/or manage at least one medical prescription of a patient, the software may transmit the information to the hospital dispensary via the at least one remote server. The information may typically be transmitted by an electronic signal. The electronic signal may be of any suitable form. The electronic signal may be a wireless signal through a wireless network (e.g., Wi-Fi (WLAN) communication, Satellite communication, RF communication, infrared communication, or Bluetooth™). For example, the electronic signal may be a GSM, CDMA, GPRS, 3G such as W-CDMA, EDGE or CDMA2000 or a 4G type electronic signal.

In some embodiments, the software may retrieve and display an estimated filling and delivery time from the hospital dispensary via the at least one remote server. The estimated filling and delivery time may typically be inputted by a pharmacist receiving the at least one medical prescription from the medical practitioner. The information may again be transmitted by an electronic signal as described above.

In some embodiments, the software may enable the medical practitioner to specify a desired recipient of the filled prescription from the hospital dispensary. For example, the medical practitioner may input that the filled prescription is to be delivered from the hospital dispensary to him or herself, another medical practitioner, a nurse or to a particular location, such as, e.g., the nurses station.

In such embodiments, the software may enable the medical practitioner to search existing staff or hospital records for a desired recipient. For example, in some embodiments, when searching and/or selecting a desired recipient, the software may retrieve relevant hospital records from the at least one remote server and display the relevant hospital records for the medical practitioner's perusal and/or selection. In some such embodiments, the medical practitioner may select a particular department of the hospital and/or station of the hospital (e.g., nurses station in cardiology department). In some such embodiments, the medical practitioner may initially narrow the hospital records by first inputting a desired recipient location and then selecting from the recipients stationed at the desired recipient location.

Again, once the medical practitioner has inputted a desired recipient or location for the filled prescription, the software may transmit the information to the hospital dispensary via the at least one remote server. The information may typically be transmitted by an electronic signal as described above.

In some embodiments, the software may further send an alert to the desired recipient alerting him or her that he or she will be receiving the filled prescription for the desired patient prescribed by the medical practitioner. The alert may be of any suitable form. For example, in some embodiments, the alert may be a page. Typically, however, the alert may be an electronic signal received on a similar data interface carried by the desired recipient.

In some embodiments, the software may require the medical practitioner to login prior to enabling the medical practitioner to at least input and/or manage at least one medical prescription of a patient.

For example, in some embodiments in which a single data interface may be shared by multiple medical practitioners, the software may require the medical practitioner to search medical practitioner records for him or herself. The software may retrieve medical practitioner records from the at least one remote server and display the records for the medical practitioner to search. In some such embodiments, the medical practitioner may select a particular department of the hospital (e.g., cardiology department). In such embodiments, the medical practitioner may narrow the medical practitioner records by first inputting the department in which he or she is located or based and then selecting him or herself from the medical practitioners listed at the particular department.

The software may preferably require the medical practitioner to input a security access number or pin prior to enabling the medical practitioner to at least input and/or manage at least one medical prescription of a patient. The security access number or pin may be of any suitable length and may include alphabetic characters, numeric characters and/or alphanumeric characters, including upper and lower case letters and/or symbols.

According to a second aspect of the present invention, there is provided a method for medical prescription management, said method including:

inputting medical prescription information for a patient;

uploading the medical prescription information for the patient to at least one remote server; and

transmitting the medical prescription information to a hospital dispensary for filling.

The method may include one or more characteristics of the system as hereinbefore described.

For example, the inputting and uploading may typically be carried out by the data interface as previously described.

The medical prescription information may include alterations to a patient's existing medical prescription or may include a new medical prescription. The medical prescription information may include the name, form and dosage for a prescribe medication. The inputting may preferably by carried out by a medical practitioner.

The method may further include delivering the filled prescription to a desired recipient. In this regard, the medical practitioner may input a name of a desired recipient when inputting the medical prescription information. The name of the desired recipient may be uploaded and transmitted to the hospital dispensary together with the medical prescription information or may be uploaded and transmitted subsequent to the medical prescription information. As previously described, the desired recipient may be the prescribing medical practitioner, another medical practitioner, a nurse or a particular location, such as, e.g., the nurses' station.

The method may include retrieving and displaying prescription drug information for the prescribed medication. The retrieving and displaying may include available stock for the prescribed drug, information about the prescribed drug, and/or dosage information.

Further advantageous features of the above aspects of the invention will be described further below.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of Invention in any way. The Detailed Description will make reference to a number of drawings as follows:

FIG. 1 illustrates a medical prescription management system according to an embodiment of the present invention;

FIG. 2 illustrates a screenshot of a start screen of an application of the system of FIG. 1, according to an embodiment of the present invention;

FIG. 3 illustrates a screenshot of a medical practitioner selection screen of the application according to an embodiment of the present invention;

FIG. 4 illustrates a screenshot of a pin input screen of the application according to an embodiment of the present invention;

FIGS. 5A and 5B respectively illustrate screenshots of patient selection screens of the application according to an embodiment of the present invention;

FIGS. 6A and 6B illustrate screenshots of a selected patient's existing prescription information screen of the application according to an embodiment of the present invention;

FIGS. 7A and 7B illustrate screenshots of a prescription input screen of the application according to an embodiment of the present invention;

FIGS. 8A and 8B respectively illustrate screenshots of an inputted prescription information screen and a confirmation of prescription screen of the application according to an embodiment of the present invention;

FIG. 9 illustrates a screenshot of a first recipient input screen of the application according to an embodiment of the present invention;

FIG. 10 illustrates a screenshot of a medical practitioner recipient screen of the application according to an embodiment of the present invention;

FIGS. 11A and 11B respectively illustrate screenshots of a nurse recipient screen of the application according to an embodiment of the present invention;

FIG. 12 illustrates a screenshot of a summary of prescription screen of the application according to an embodiment of the present invention; and

FIG. 13 illustrates a prescription management method according to an embodiment of the present invention.

### DETAILED DESCRIPTION

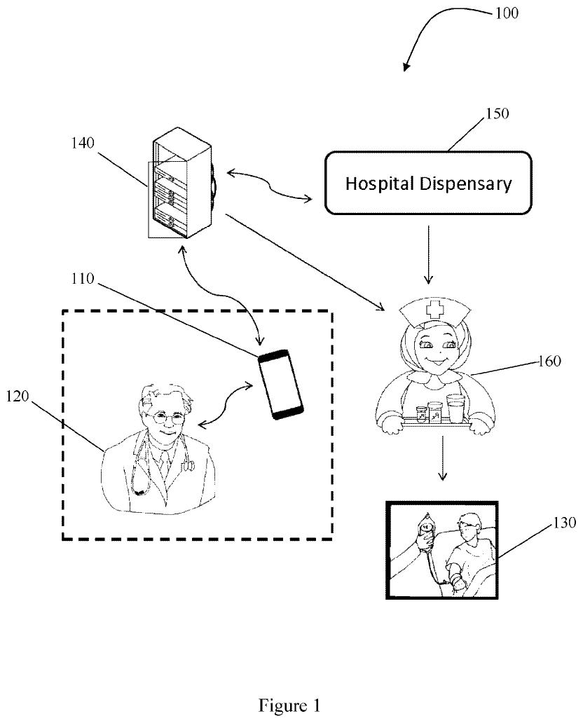

FIG. 1 illustrates a prescription management system (100) according to an embodiment of the present invention. The system (100) is particularly suited for a hospital environment.

The system (100) includes an application (app; i.e., software) configured to run on a mobile device (110; i.e., data interface) of a medical practitioner (120). The app is interactive enabling the medical practitioner (120) to interact with the system (100) to at least input and/or manage at least one medical prescription of a patient (130). The system (100) further includes a remote server (140) in communication with the app for at least receiving the at least one medical prescription of the patient (130) and transmitting the at least one medical prescription to a hospital dispensary (150) for filling and delivery to a desired recipient (160).

The app provides a user interface through which the medical practitioner (120) can enter data.

The app can be installed on the mobile device (110) or be downloaded on demand.

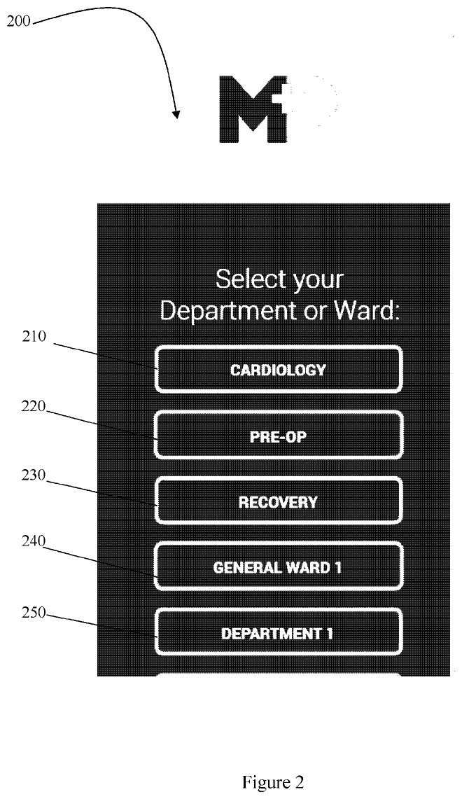

FIG. 2 illustrates a screenshot (200) of a start screen of the app of the system. The start screen is provided in response to the medical practitioner launching the app.

As shown, the start screen includes a menu in the form of a cardiology button (210), a pre-op button (220), a recovery button (230), a general ward 1 button (240) and a department 1 button (250).

In use the medical practitioner selects the button corresponding to the particular location of him or herself.

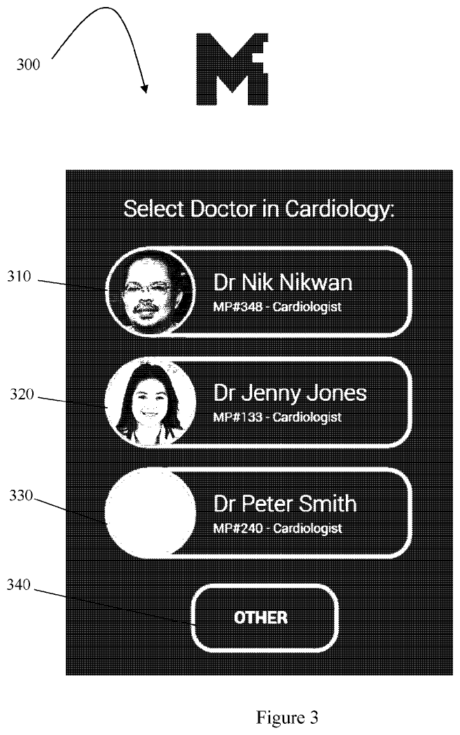

FIG. 3 illustrates a screenshot (300) of a medical practitioner selection screen of the app of the system for the medical practitioners present in the location selected in FIG. 2.

As shown, the screen again includes a menu in the form of a first button (310) corresponding to a first medical practitioner present in the cardiology ward, a second button (320) corresponding to a second medical practitioner present in the cardiology ward, a third button (330) corresponding to a third medical practitioner present in the cardiology ward, and a fourth button (340) enabling the medical practitioner to input another medical practitioner not listed.

In use, the medical practitioner selects the button corresponding to him or herself.

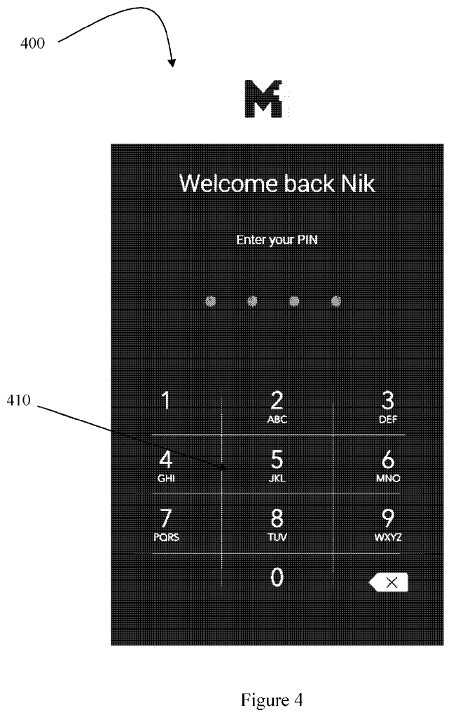

FIG. 4 illustrates a screenshot (400) of pin input screen of the app of the system.

As shown, the pin input screen presents a numerical buttons (410) enabling the medical practitioner to input his or her pin and obtain permission to interact with the system.

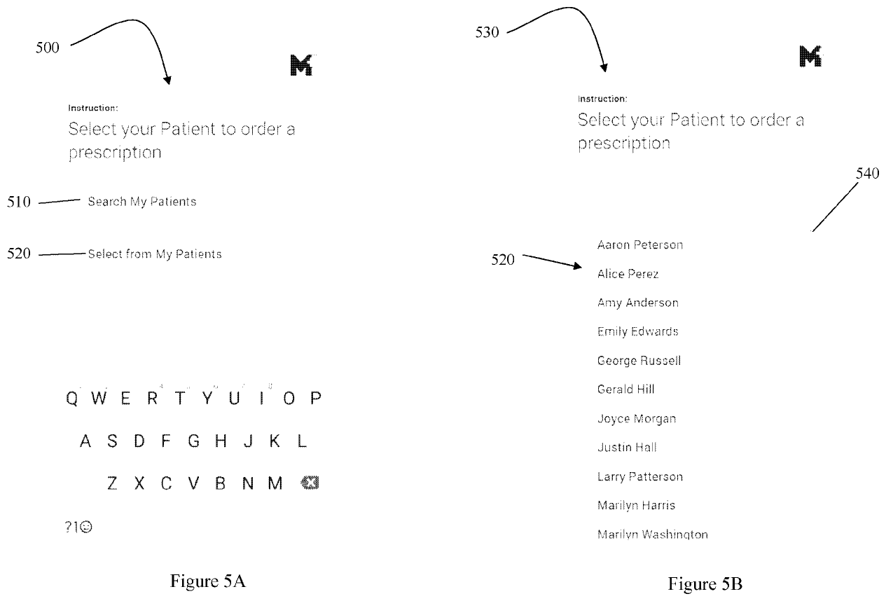

FIG. 5A illustrates a screenshot (500) of a patient selection screen of the app of the system.

As shown, the patient selection screen presents a patient search box (510) enabling the medical practitioner to enter the name of the desired patient and a patient drop down box (520) enabling the medical practitioner to select the desired patient from a list of his or her patients.

FIG. 5B illustrates a screenshot (530) of the patient drop down box (520) of the app of the system being used. The drop down box (520) provides scroll cursors (540) for the medical practitioner to scroll through the list.

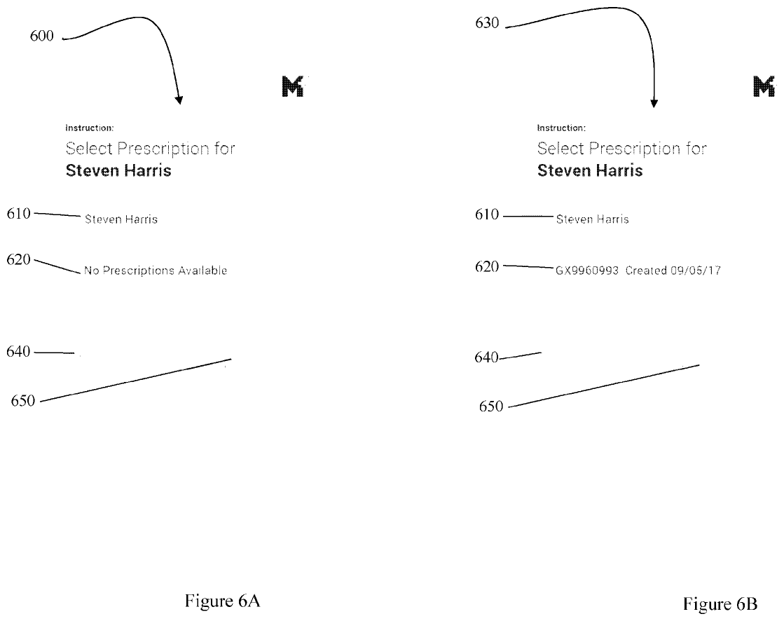

FIG. 6A illustrates a screenshot (600) of an existing prescription information screen of the app of the system for a selected patient.

As shown, the existing prescription information screen presents the selected patient's name (610) and an existing prescriptions box (620). In this figure, the patient “Steven Harris” is shown as having no existing prescriptions.

FIG. 6B illustrates an alternative screenshot (630) to screenshot (600) shown in FIG. 6A. In this screenshot (630), the screen again presents the selected patient's name (610) and an existing prescriptions box (620). However, in this figure, the patient “Steven Harris” is shown as having an existing prescription.

In both FIGS. 6A and 6B, the screenshots (600, 630) additionally present a “NEW PRESCRIPTION” button (640) and a “NEXT” button (650).

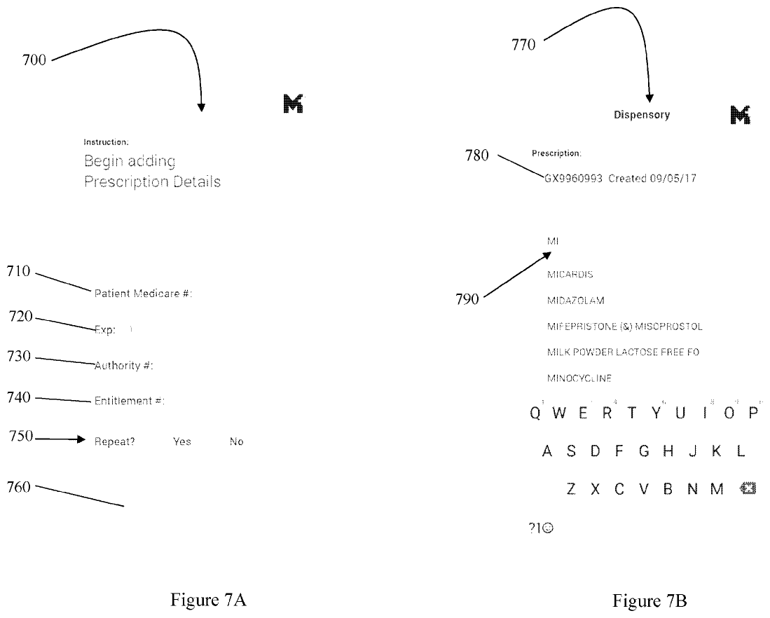

FIG. 7A illustrates a screenshot (700) of a prescription input screen of the app of the system enabling the medical practitioner to input a new medical prescription for the selected patient.

As shown, the screen includes a number of input boxes, including a patient medicare number input box (710), a medicare card expiry input box (720), an authority input number box (730) and an entitlement input number box (740). The screen additionally presents repeat required “yes” or “no” radio toggle boxes (750) and a “START PRESCRIPTION” button (760).

FIG. 7B illustrates a screenshot (770) of a subsequent prescription input screen of the app of the system in which the medical practitioner can select a desired prescription medicine.

As shown, the screen lists existing prescriptions in a drop down list (780) at the top of the screen and provides an input and search input box (790) below drop down list (780) for the medical practitioner to search for and select a desired prescription medicine.

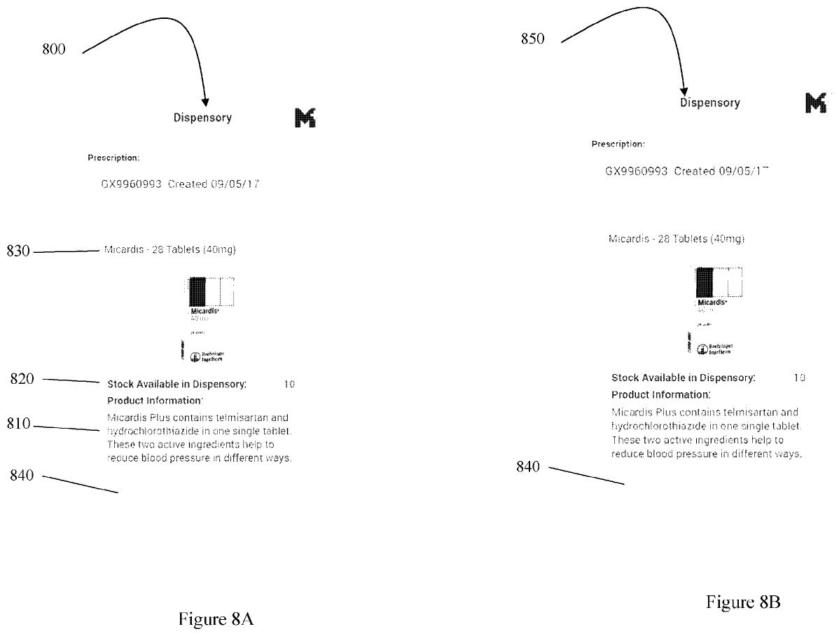

FIG. 8A illustrates a screenshot (800) of an inputted prescription information screen of the app of the system.

As shown, the screen again lists existing prescriptions in a drop down list (780) at the top of the screen and provides a brief description (810) of the selected prescription medicine together with a quantity in stock number (820), a drop down list (830) for the medical practitioner to select a desired dosage for the selected prescription medicine and a “ADD TO DISPENSARY” button (840).

FIG. 8B illustrates a screenshot (850) of the same screen as shown in FIG. 8B save that the medical practitioner has confirmed the order by pressing the “ADD TO DISPENSARY” button (840).

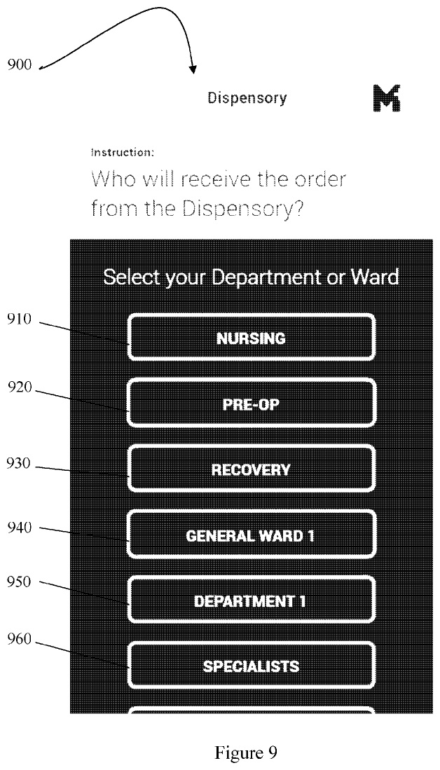

FIG. 9 illustrates a screenshot (900) of a first recipient input screen of the app of the system in which the medical practitioner can select a desired recipient to receive the filled prescription from the hospital dispensary.

As shown, the screen includes a menu in the form of a NURSING button (910), a PRE-OP button (920), a RECOVERY button (930), a GENERAL WARD 1 button (940), a DEPARTMENT 1 button (950) and a SPECIALISTS button (960).

In use, the medical practitioner selects the button corresponding to the location where the desired recipient is based or located.

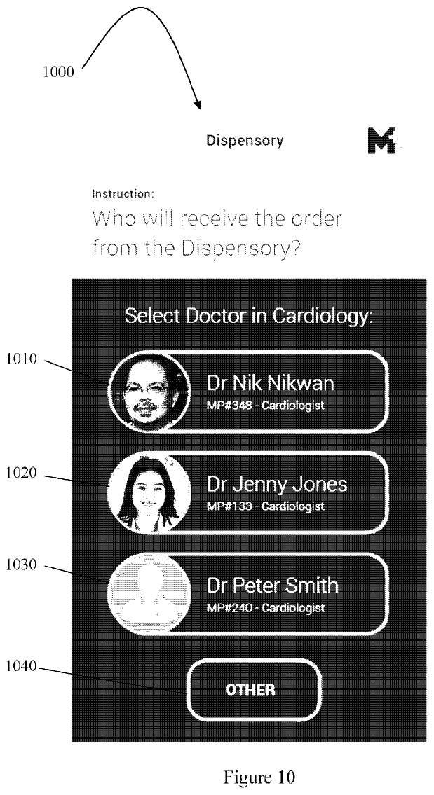

FIG. 10 illustrates a screenshot (1000) of a medical practitioner recipient screen of the app of the system in which the medical practitioner can select him or herself or another medical practitioner in the same ward or department to receive the filled prescription.

As shown, the screen provides a menu in the form of a first button (1010) corresponding to the medical practitioner, a second button (1020) corresponding to another medical practitioner, a third button (1030) corresponding to yet another medical practitioner, and an “OTHER” button (1040) enabling the medical practitioner to search for and select yet another desired medical practitioner recipient.

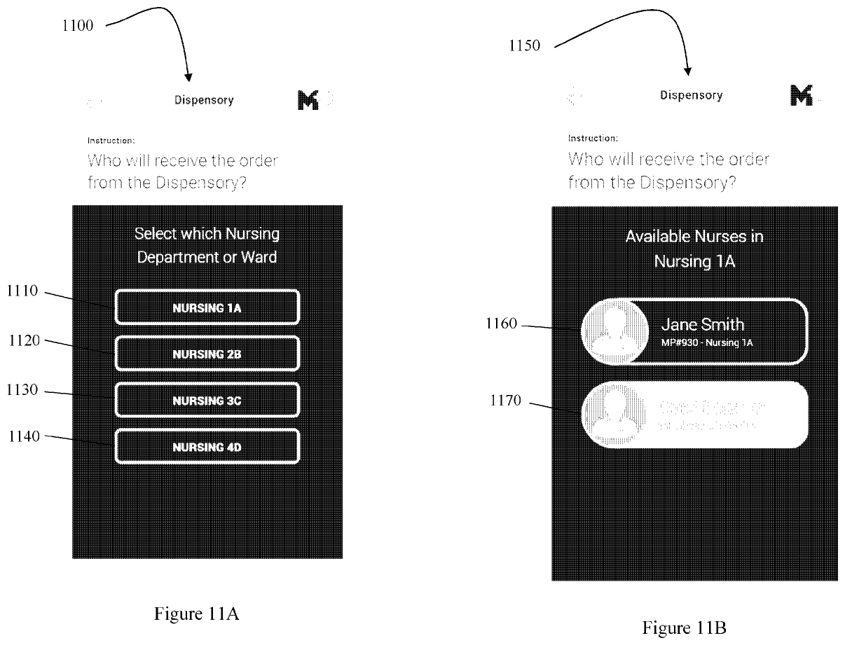

FIG. 11A illustrates a screenshot (1100) of a nurse recipient screen of the app of the system in which the medical practitioner can select a nursing department or ward containing a desired nurse recipient.

As shown, the screen present a menu including a NURSING 1A button (1110), a NURSING 2B button (1120), a NURSING 3C button (1130) and a NURSING 4D button (1140).

In use, the medical practitioner selects the button corresponding to the department or ward in which the desired nurse recipient is based or located.

FIG. 11B illustrates a screenshot (1150) of a subsequent nurse recipient screen of the app of the system in which the medical practitioner can select a desired nurse recipient in the NURSING 1A department or ward selected in the previous screen.

As shown, the screen presents a menu including a first button (1160) corresponding to nurse Jane Smith and a second button (1170) corresponding to nurse Carol Stevens.

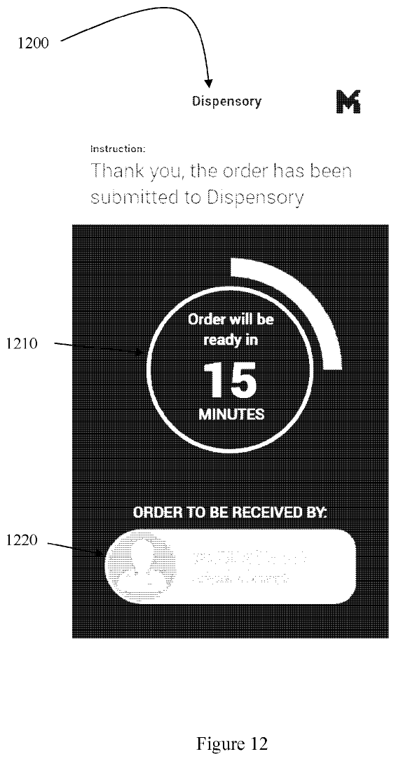

FIG. 12 illustrates a screenshot (1200) of a summary of prescription screen of the app of the system.

As shown, the screen provides an estimated order to be filled time (1210) and a summary (1220) of the name and location of the selected desired recipient.

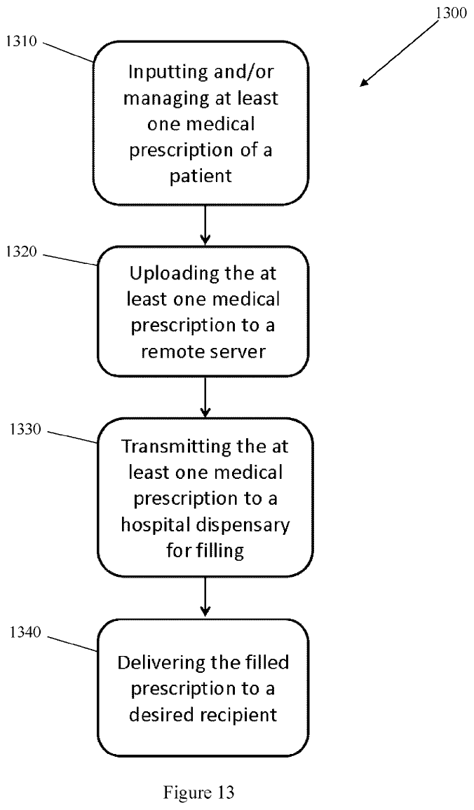

A method (1300) of using the system (100) as shown in FIG. 1 to input and/or manage at least one medical prescription of a patient (130) is now described with reference to FIG. 13.

At step 1310, the medical practitioner (120) utilises the mobile device (110) running the app to input and/or manage at least one prescription for the patient (130). The medical practitioner (120) optionally inputs in the app that the desired recipient (160) is to receive the medical prescription when filled.

At step 1320, the mobile device (110) running the app uploads the at least one prescription to a remote server (140).

At step 1330, the remote server (140) transmits the at least one medical prescription to the hospital dispensary (150) for filling.

At step 1340, the hospital dispensary delivers the filled prescription to the selected desired recipient (160).

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A system for medical prescription management, said system including:
a data interface through which a medical practitioner can interact with the system to at least input and/or manage at least one medical prescription of a patient;
at least one remote server for at least receiving the at least one medical prescription of the patient from the data interface and transmitting the at least one medical prescription to a hospital dispensary for filling; and
software in the form of an application configured to be run on a user device having a display upon which the data interface is generated and displayed, wherein once the medical practitioner has interacted with the system to at least input and/or manage the at least one medical prescription of a patient, the software transmits the information to the hospital dispensary via the at least one remote server, and
wherein the software retrieves and displays an estimated filling and delivery time from the hospital dispensary via the at least one remote server.

2. A system for medical prescription management as claimed in claim 1 wherein the data interface is configured to enable the medical practitioner to interact with the system and at least input and/or manage the at least one medical prescription of a patient.

3. A system for medical prescription management as claimed in claim 1 wherein the data interface includes a user interface through which the medical practitioner enters data, and/or recipient details.

4. A system for medical prescription management as claimed in claim 1 wherein the data interface is in the form of an external processing device, including one or more processors and a memory, for receiving input from the medical practitioner and transmitting and receiving information to and from the at least one remote server.

5. A system for medical prescription management as claimed in claim 1 wherein the data interface is configured to be hand-held by the medical practitioner.

6. A system for medical prescription management as claimed in claim 1 wherein the software enables the medical practitioner to search existing patients for a desired patient.

7. A system for medical prescription management as claimed in claim 6 wherein when searching and/or selecting a desired patient, the software retrieves relevant patient records from the at least one remote server and display the relevant patient records for the medical practitioner's perusal and/or selection.

8. A system for medical prescription management as claimed in claim 6 wherein the software retrieves and display the selected patient's details, including but not limited to, age, gender, address details, healthcare system details and/or private healthcare details.

9. A system for medical prescription management as claimed in claim 6 wherein the software retrieves and displays a selected patient's existing prescription(s) and/or enable the medical practitioner to manage the selected patient's existing prescription(s).

10. A system for medical prescription management as claimed in claim 6 wherein the software enables the medical practitioner to input a new prescription.

11. A system for medical prescription management as claimed in claim 1 wherein the software enables the medical practitioner to specify a desired recipient of the filled prescription from the hospital dispensary.

12. A system for medical prescription management as claimed in claim 11 wherein once the medical practitioner has inputted a desired recipient or location for the filled prescription, the software transmits the information to the hospital dispensary via the at least one remote server.

13. A system for medical prescription management as claimed in claim 11 wherein the software sends an alert to the desired recipient alerting him or her that he or she will be receiving the filled prescription for the desired patient prescribed by the medical practitioner.

14. A system for medical prescription management as claimed in claim 1 wherein a single data interface is shared by multiple medical practitioners, and the data interface requires the medical practitioner to search medical practitioner records for him or herself and retrieve medical practitioner records from the at least one remote server and display the records for the medical practitioner to search.

15. A system for medical prescription management as claimed in claim 14 wherein the data interface requires the medical practitioner to input a security access number or pin for authorisation purposes prior to enabling the medical practitioner to at least input and/or manage at least one medical prescription of a patient.

16. A method for medical prescription management, said method including:
inputting medical prescription information for a patient into a data interface;
uploading, using the data interface, the medical prescription information for the patient to at least one remote server; and
transmitting, using software configured to be run on a user device having a display on which the data interface is generated and displayed, the medical prescription information to a hospital dispensary for filling, and
retrieving using the software, and displaying on the display, an estimated filling and delivery time from the hospital dispensary.

17. A method for medical prescription management as claimed in claim 16 wherein the medical prescription information includes alterations to a patient's existing medical prescription or may include a new medical prescription.

18. A method for medical prescription management as claimed in claim 16 further including delivering the filled prescription to a desired recipient.

19. A method for medical prescription management as claimed in claim 18 wherein the name of the desired recipient is uploaded and transmitted to the hospital dispensary together with the medical prescription information.

20-23. (canceled)
